(19) United States
(12) Patent Application Publication
(10) Pub. No.: US 2004/0185032 A1
(43) Pub. Date: Sep. 23, 2004

(54) Compositions and methods for treating colic

(76) Inventor: David Burrell, Wake Forest, NC (US)

Correspondence Address: MCDERMOTT, WILL & EMERY, 600 13th Street, N.W., Washington, DC, 20005-3096, US

(21) Appl. No.: 10/689,539

(22) Filed: Oct. 21, 2003

Related U.S. Application Data
(60) Provisional application No. 60/455,417, filed on Mar. 18, 2003.

(51) Int. Cl. A61K 45/00

(52) U.S. Cl. 424/93.45

(57) ABSTRACT

Compositions and methods for providing relief from pain and/or discomfort associated with gastrointestinal disorders, including, for example, bloating, crying, gas, cramping, regurgitation, diarrhea and gastrointestinal pain, associated with colic comprising, at least one antiflatulent, at least one histamine H₁-receptor antagonist, and optionally, one or more prebiotic and/or one or more probiotic.

This application claims the benefit of U.S. Provisional Application No. 60/455,417, filed on Mar. 18, 2003, which is incorporated herein by reference.

### FIELD OF INVENTION

This invention relates to compositions and methods of treatment for gastrointestinal disorders. More particularly, the invention relates to compositions and methods for providing relief from pain and/or discomfort associated with colic. It also relates to compositions and methods for providing relief from pain and/or discomfort associated with inflammatory bowel disease, and infectious diarrhea.

### BACKGROUD OF THE INVENTION

Colic is a self-limiting, developmental gastrointestinal disorder that affects approximately twenty percent (20%) of all infants. While the etiology of colic is not completely understood, medical experts believe its symptom logy may be linked to an immature digestive system, allergies, hormones in breast milk, and overfeeding. Colic is a symptom complex characterized by paroxysms of presumably sever abdominal pain, caused by bloating, gas, cramping, regurgitation, diarrhea and gastrointestinal pain, and crying with irritability and fussing in an otherwise healthy infant. Episodes of colic tend to be worse in the evenings and do not respond to the usual means of comforting, such as feeding, cuddling or diaper changing. Colic equally affects boys and girls, first-born children and those born later. It often first appears around two to four weeks of age and can last for three months, or longer.

An infant suffering from colic typically presents with prolonged crying and inability to rest, resulting in hyperirritability and stress, both on the infant as well as caregivers. Infantile colic is considered to be one of the most frequent reasons parents seek medical attention for children during the first weeks of life. Despite the benign and self-limiting course, infantile colic can impose a substantial psychological, emotional and physical burden for parents. Colic can interfere with parent bonding, cause strain in a marriage, lead to unnecessary hospitalizations, and in some unfortunate cases child abuse. (Balan, A. J., Management of infantile colic Amer Pham Physician 1997;55:235-241). Mothers of babies with colic may experience fantasies of infanticide. (Levitsky, S. et al., Infant colic syndrome-maternal fantasies of aggression and infanticide Clin Pediatr 2000;39:395-400). Moreover, colic does not always disappear without a trace, and some children, who suffered from colic in infancy, may experience gastrointestinal problems later in their lives. (Iacano, G., et al., Severe infantile colic and food intolerance: a long-term prospective study. J Pediatr Gastroenterol Nutr 1991;12:332-5). Pediatricians are challenged to seek therapies that prove effective in treatment of colic and provide relief, both for infant as well as caregivers.

At present there is no cure for colic. The current treatment paradigm for colic consists of either pharmacological and/or non-pharmacological methods, providing at best marginal reduction of symptoms. Typical therapeutic interventions for colic offered to parents fall within four categories, including, dietary, physical, behavioral and pharmacological.

Dietary manipulations include professional advice on various feeding techniques, or the use of hypoallergenic milk, soy or lactose free formulas, and an early introduction to solids. (Lothe, L., et al. cow's milk formula as a cause of infantile colic: a double-blind study. Pediatrics 1982;70:7-10; Forsyth B W C. Colic and the effect of changing formulas: a double-blind multiple-crossover study. J Pediatr 1989;115,521-6; Treem, W R, et al. Evaluation of the effect of a fiber-enriched formula on infant colic. J Pediatr 1991; 119695-701). However, neither the use of soy formulas, or changes in feeding techniques works effectively for every case of colic. A review of the data studying these recommendations showed that use of hypoallergenic formula, such as partially hydrolyzed or amino acid-based, may benefit only about 25% of infants. (Lucassen, P L B J, et al. Infantile colic: crying time reduction with a whey hydrolysate: a double-blind, randomized placebo-controlled trial. Pediatrics 2000;106:1349-54; Estep, D C, et al. Treatment of infant colic with amino acid-based infant formula: a preliminary study. Acta Paediatr 2000;89:22-7).

Physical strategies for the management of colic include physical movement of body positions to alleviate gas production/reflux, carrying, swaddling, applying abdominal pressure, or massaging the baby. Other methods include creating a sense of distraction to minimize infant awareness of colic such as taking an infant for a car ride, use of a car ride simulator, crib vibrator, or infant swings (Lipton E L. Swaddling and child care practice: historical, cultural and experimental observations. Pediatrics 1965;35:521-67; Byrne J M, Horowitz F D. Rocking as a soothing intervention: the influence of direction and type of movement. Infant Behav Dev 1981;4:207-18). Another approach is to play recordings of sounds that supposedly soothe the baby. However, there is evidence in the medical literature that these methods do not work (Parkin P C, Schwartz C J, Manuel B A. Randomized controlled trial of three interventions in the management of persistent crying of infancy. Pediatrics 1993;92(2): 197-201). These strategies, at best, are only marginally effective in abatement of colic symptoms.

Recommendations for behavioral interventions for the treatment of colic are the most inconsistent therapies available. Some authors advocate increasing sensory stimulation, while others advocate decreasing such stimulation (Balon A J. Management of infantile colic. Amer Pham Physician 1997;55:235-242; Lucassen P L B J, Assendelft W J J, Gubbels J W, van Eijk T M, van Geldrop W J, Effectiveness of treatments for infantile colic: systematic review. BMJ 1998;316(5): 1563-9; and Carey W B, “Colic”—primary excessive crying as an infant-environmental interaction. Pediatr Clin North Am 1984;31:993-1005). Other recommendations include early response to crying, or allowing the infant to cry, offering a pacifier, implementation of a routine feeding schedule, using eye contact and interactive playing.

Pharmacologic intervention for the treatment of colic has led to the use of prescription and non-prescription medications. Currently employed prescription medications include belladonna alkaloids and opiates (paregoric), which may provide relief, but are fraught with risks including extra pyramidal symptoms, respiratory depression, and constipation. For example, anticholinergic drugs, similar in their effect to atropine, such as, Hyoscyamine (LEVISINE™, or GASTROSED™) and Dicyclomine dilate pupils, increase heart rate, decrease production of saliva, relieve spasms of gastrointestinal and urinary tracts, as well as bronchi. Although the anticholinergic drugs are the only prescription medications on the U.S. market that consistently have been shown to effectively treat infantile colic, unfortunately, up to 5% of treated infants may develop side effects, including breathing difficulties, apnea, seizures, syncope, asphyxia, coma and muscular hypotonia (Williams J, Watkin-Jones R. Dicyclomine: worrying symptoms associated with its use in some small babies. BMJ 1984;288:901; Myers J H, Moro-Sutherland D, Shook J E. Anticholinergic poisoning in colicky infants treated with hyoscyamine sulfate. Am J Emerg Med 1997;15:532-5). In addition, several cases of death have been reported in infants taking dicyclomine (Garriott J C, Rodriguez R, Norton L E. Two cases of death involving dicyclomine in infants. Clinical Toxicol 1984;22(5):455-462).

Non-prescription medications that have been reported as effective treatment for infantile colic include several sedative or sleep-inducing drugs, including supraphysiologic (high dose) diphenhydramine (BENADRYL®), phenobarbital, chloral hydrate, and even alcohol. However, there is the potential for serious side effects associated with several of these agents in children with respiratory disease, thus limiting their widespread use in treating colic (Balon A J. Management of infantile colic. Amer Pham Physician 1997;55:235-242; Gurry D. Infantile colic. Australian Pham Phys 1994;23(3):337-34632).

A safer non-prescription medication for treatment of colic has largely included the administration of simethicone or dimethylpolysiloxane, a non-absorbable, over-the-counter drug, which reduces the size of intestinal gas bubbles. Simethicone has a very safe profile and is frequently recommended, despite several studies demonstrating that effectiveness of simethicone on infantile colic is no better than placebo. (Metcalf, T J, et al., Pediatrics 1994 July; 94(1):29-34. Sferra, T J, et al., Pediatr Clin North Am 1996 April; 43(2):489-510. Danielson, B. et al., Acta Paediatr Scand 1985 May; 74(3):446-50. Colon, A R, et al., Am Fam Physician 1989 December; 40(6):122-4.). As a result, the most common treatment for colic today is to simply wait for the baby to grow out of the condition.

Therefore, there currently is a need for safe and effective pharmacologic compounds and compositions and non-pharmacologic techniques that prove useful for treating colic in infants and young children. The compositions and methods of the present invention, respond to this need, providing a pharmacologic combination product (PROMETHADRYL® that can safely and effectively treat the multiple symptoms associated with colic in infants and young children.

Inflammatory bowel disease (IBD) is a general term for a group of diseases involving gut-wall inflammation. Chronic IBD is generally divided into two major groups: Chrohn's disease and ulcerative colitis. Although some significant differences exist in their location and the way they affect the bowel wall, they both cause abdominal pain and cramping with frequent, urgent, loose bowel movements marked by blood, mucus, and pus. Complications of both can include abscesses and infections, fitulas, hemorrhoids, intestinal wall perforations, malabsorption of nutrients, and weight loss. IBD in general can increase the risk for gastrointestinal cancer. Additionally, the disease can have systemic effects including arthritic symptoms and fatigue. IBD can be a chronic, relapsing, and debilitating condition. Many patients with IBD face the possibility of long-term drug use with significant side effects (steroids, immunosuppressive drugs, and salicylic acid derivatives such as sulfasalazine and mesalamine) such as frequent infections, anemia, easy bruising, and mood swings. Thus, many people prefer the disease symptoms to the side effects. Research (Munkholm, Gut 1994;35) suggests impaired musosal gut barrier malfunction or impairment can lead to a variety of diseases, among them IBD. The link between IBD and compromised intestinal integrity is clear. Therefore, the need for bacterial gut balance and decreasing toxic load are of great interest in the field of IBD research.

Since there are more than 400 known bacterial species residing in the human gastrointestinal tract, their overall balance can profoundly affect gut ecology and health. Research has shown bacterial are capable of producing toxins and antitoxins, altering chemical composition of drugs and food, can produce and degrade vitamins, degrade dietary toxins, and inhibit the growth of certain pathogens. Gut-derived products may also play a role in increasing the systemic immune inflammatory response (Alexander Ann Surg 1990;212 (4)). Studies with animals (Pirzer Lancet 1991;338) with experimentally induced IBD support the idea that resident bacteria—and not pathogenic toxins, are the reason for IBD. Studies (Giaffer J Med Microbiol 1991;35) with patients with active Chrohn's disease demonstrated a markedly different intestinal flora as compared to patients with quiescent disease, ulcerative colitis, or normal controls. In these individuals, the concentration of aerobic bacteria was elevated, especially Escherichia coli, as well as anerobic bacteria Bacteriodes fragilis and Bacteriodes vulgatus. Additionally, in all patients with Chrohn's disease, Bifidobacteria were decreased.

In another (Gionchetti Gastroenterology 1998;114) double-blind, placebo-controlled study 40 patients with ulcerative colitis were treated with either a special probiotic preparation of B. longum, B. infantis, B. brevis, L acidophilus, L. casei, L. delbruekii, L. plantarum, and S. Thermophilus or placebo. After six (6) months, 85% (17/20) of the probiotic-treated patients were asymptomatic, whereas 100% (20) of the placebo group relapsed.

Thus, the product PROMETHADRYL® which contains Simethicone for treatment of flatulence/bloating, diphenhydramine for treatment/symptoms of cramping/nausea, arabinogalactans (larch burch) as a fiber source and prebiotic substrate for beneficial bacterial species Bifidobacteria and Lactobacilli, plus probiotic compounds would be useful in treatment of symptoms associated with IBD (PROMETHADRYL® PLUS).

Infectious diarrhea is a worldwide health problem. In many developing countries, diarrheal disease remains a leading cause of death and illness among infants and children (Snyder Bull World Health Organ 1982;60, Ho JAMA 1988;260). A number of probiotics have been used with varying success to prevent diarrheal disease. Therefore, using selective beneficial probiotic substrate, plus the addition of prebiotic substrate, such as arabinogalactans or inulin, to help promote beneficial bacteria (Bifidibacteria and Lactobacillus) and Simethicone (gas/bloating) and Diphenhyramine (cramping/nausea), the product PROMETHADRYL® PLUS is beneficial in treatment of the symptoms associated with infectious diarrhea (including traveller's diarrhea).

### SUMMARY OF THE INVENTION

The present invention provides compositions and methods that are useful for treating gastrointestinal disorders, including without limitation, acid indigestion, colic, diarrhea, heartburn, irritable bowel syndrome, sour stomach, gas associated with the foregoing conditions, gastric ulcers, peptic ulcer disease of the esophagus, stomach or duodeum, indigestion, flatulence, dyspepsia of unknown origin including cancer of the stomach, infiltrative disease of the stomach including lymphoma, Crohn's disease, eosinophilic granuloma, tuberculosis, syphilis and sarcoidosis, abdominal lesions, chronic pancreatis, bilary disease, Zollinger-Ellison syndrome, motion sickness, otitis media and other diseases and conditions of the gastro-intestinal tract.

The present invention further provides compositions and methods that are useful for the treatment of immune disorders, for example, diabetes, cancer and HIV. The introduction of one or more prebiotic substrate, such as, larch arabinogalactans or inulin, promotes enhancement of the immune system in immunosuppressed individuals (e.g. diabetic, cancer, and HIV patients), reduction of symptoms associated with inflammatory bowel disease (IBD), inflammatory bowel syndrome (IBS), reduction of immune-related disorders such as allergies and recurrent infectious diarrhea, increased medium chain fatty acid production, and folate (vitamin) synthesis through enhanced development of prebiotic gastrointestinal ecology. This may prove especially beneficial in patients with co-morbid diseases which exhibit problems secondary to impaired gastrointestinal health.

Generally, the pharmaceutical compositions comprise an effective amount of at least one antiflatulent, at least one histamine H₁-receptor antagonist and optionally, one or more prebiotic such as larch arabinogalactans or inulin, and/or one or more probiotic, and the methods involve administering together or substantially together at least one antiflatulent, at least one histamine H₁-receptor antagonist, and optionally, one or more prebiotic such as, larch arabinogalactans or inulin, and/or one or more probiotic at or after the onset of pain and/or discomfort, associated with gastrointestinal disorders to a subject in need thereof. Preferably, the compositions and methods are for the treatment of colic, irritable bowel syndrome, indigestion, dyspepsia and flatulence.

In a preferred embodiment, the present invention provides compositions and methods, which provide relief from the pain and/or discomfort associated with colic in a subject in need thereof. Generally, the pharmaceutical compositions include an effective amount of at least one antiflatuent and at least one histamine H₁-receptor antagonist, and optionally, one or more prebiotic and/or one or more probiotic, and the methods involve administering together or substantially together at least one antiflatulent, at least one histamine H₁-receptor antagonist, and optionally one or more prebiotic and/or one or more probiotic at or after the onset of pain and/or discomfort, including, relief from pain and/or discomfort, caused by, for example, bloating, crying, gas, cramping, regurgitation, diarrhea and gastrointestinal pain, associated with colic to a subject in need thereof.

The present invention is based upon the discovery that the combination of antiflatulents, histamine H₁-receptor antagonists, and optionally, larch arabinogalactans and/or one or more probiotic can be effectively administered together or substantially together for enhanced therapeutic treatment of gastrointestinal disorders, including, preferably, relief from the pain and/or discomfort, caused by, for example, bloating, crying, gas, cramping, regurgitation, diarrhea and gastrointestinal pain, associated with colic. It is surprising that the combination of an antiflatulent and a histamine H₁-receptor antagonist provides an effective result better than using either feature alone.

In one embodiment of the invention there are pharmaceutical compositions for the treatment of colic comprising simethicone and diphenhydramine. Preferably, the composition comprises larch arabinogalactans and/or one or more probiotic. Preferably, the compositions are for the treatment of colic which provide relief from pain and/or discomfort, caused by, for example, bloating, crying, gas, cramping, regurgitation, diarrhea and gastrointestinal pain, associated with colic comprising, in admixture with a pharmaceutically acceptable carrier, an antiflatulent, a competitive histamine H₁-receptor antagonist, and larch arabinogalactans.

More preferably, the compositions comprise an antiflatulent in an amount effective to substantially change the surface tension of gas bubbles. Most preferably, the compositions comprise simethicone as an antiflatulent.

It is also preferable that the compositions comprise at least one competitive histamine H₁-receptor antagonist in an amount to substantially inhibit respiratory, vascular and gastrointestinal smooth muscle constriction. Preferably, a histamine H₁-receptor antagonist provides antimuscarinic activity (reduce GI cramping), and anticholinergic activity (reduces nausea and promotes sedation). Most preferably, the compositions comprise diphenhydramine as a competitive histamine H₁-receptor antagonist.

It is also preferable that the compositions comprise one or more prebiotic in an amount to increase immune-modulating activity in the gastro-intestinal tract. Most preferably, the compositions comprise larch arabinogalactans and/or inulin.

It is also preferable that the compositions comprise one or more probiotic in an amount to encourage healthy gut ecology and normal gastrointestinal function.

Preferably, the pharmaceutical compositions for the treatment of colic are in oral dosage forms, more preferably a liquid dosage form such as a suspension.

It is also preferable that the compositions are substantially free of dyes, alcohols, artificial sweeteners, such as saccharin and aspartamine, artificial flavors and artificial preservatives In another aspect of the invention there are methods for the treatment of colic comprising administering to a subject in need thereof, a therapeutically effective dose of a composition comprising at least one antiflatulent, at least one histamine H₁-receptor antagonist, and optionally, one or more prebiotic, such as, larch arabinogalactans or inulin, and/or one or more probiotic. Preferably the antiflatulent, histamine H₁-receptor antagonist, prebiotic and probiotic are selected on a basis for therapeutic treatment of colic.

At least one of the above aspects/embodiments and advantages may be realized and attained by means of the instrumentalies and combinations particularly recited in the appended claims and/or supported by this written description. Additional aspects/embodiments and attendant advantages of the present invention will be set forth, in part, in the description that follows, or may be learned from practicing or using the present invention. It is to be understood that the foregoing general description and the following detailed description are exemplary and explanatory only and are not to be viewed as being restrictive of the invention, as claimed.

### DETAILED DESCRIPTION OF THE INVENTION

All patents, patent applications and literature cited in this description are incorporated herein by reference in their entirety. In the case of inconsistencies, the present disclosure, including definitions, will prevail.

In a preferred embodiment, the present invention provides novel pharmaceutical compositions and methods for the treatment of gastrointestinal disorders, such as colic, comprising at least one antiflatulent and at least one histamine H₁-receptor antagonist in admixture with a pharmaceutically acceptable carrier. In a further preferred embodiment, the present invention is directed to novel pharmaceutical compositions and methods for the treatment of gastrointestinal disorders, such as colic, comprising at least one antiflatulent, at least one histamine H₁-receptor antagonist and one or more prebiotic such as, larch arabinogalactans and/or inulin, in admixture with a pharmaceutically acceptable carrier. It is also preferred that these compositions and methods provide relief from the pain and/or discomfort, caused by, for example, bloating, crying, gas, cramping, regurgitation, diarrhea and gastrointestinal pain associated with gastrointestinal disorders, and in particular colic.

As used herein, the term “antiflatulent” refers to a substance that reduces an amount of flatus from the stomach and intestines. Non-limiting exemplary antiflatulents or derivatives thereof contemplated by the present invention include, maltodextrin, and oraganopolysiloxanes such as, dimethylpolysiloxane, methylpolysiloxane and simethicone.

As used herein, the term “colic” refers to the symptom complex characterized by paroxysms of presumably severe abdominal pain, caused by, for example, bloating, gas, cramping, regurgitation, diarrhea, and/or gastrointestinal pain, and crying with irritability and fussing in an otherwise healthy infant or young child.

As used herein, the term “gastrointestinal disorder” refers to pain and symptoms associated with a wide variety of gastrointestinal ailments and conditions including diseases or conditions such as, for example, acid indigestion, colic, diarrhea, heartburn, irritable bowel syndrome, sour stomach, gas associated with the foregoing conditions, peptic ulcer disease of the esophagus, stomach or duodeum, indigestion, flatulence, dyspepsia of unknown origin including cancer of the stomach, infiltrative disease of the stomach including lymphoma, Crohn's disease, eosinophilic granuloma, tuberculosis, syphilis and sarcoidosis, abdominal lesions, chronic pancreatis, bilary disease, Zollinger-Ellison syndrome, motion sickness, otitis media and other diseases and conditions of the gastro-intestinal tract.

As used herein, the term “histamine H₁-receptor antagonist” refers to a substance which exerts a pharmacologic action by specifically blocking the H₁ histamine receptors. Antihistamines are reversible competitive H₁-receptor antagonists which reduce or prevent most of the physiologic effects that histamines normally produce, including inhibition of respiratory, vascular, and gastrointestinal smooth muscle constriction. Exemplary histamine H₁-receptor antagonists contemplated by the present invention include, without limitation, acrivastine, astemizole, azatadine, azclastine, bromodiphenhydramine, brompheniramine, cetirizine, chlorpheniramine, clematine, cyproheptatine, desloratadine, dexbrompheniramine, dexchlorpheniramine, diphehydramine, doxylamine, fexofenadine, hydroxyzine, ketoffen, loratidine, norastemizole, phenindamine, pyrilamine, temelastine, terfenadine, tripelennamine, triprolidine and pharmaceutically effective derivatives and salts thereof.

As used herein, the term “pain and/or discomfort” refers to an unpleasant sensation or state of being associated with actual or potential bodily disorder (such as a disease or injury). Some of the symptoms of pain and/or discomfort include without limitation, bloating, crying, gas, cramping, reflux, regurgitation diarrhea and gastrointestinal pain.

The term “pharmaceutically acceptable carrier or adjuvant” refers to a carrier or adjuvant that may be administered to a subject, together with an antiflatulent, histamine H₁-receptor antagonist, and optionally, larch arabinogalactans and/or one or more probiotic of the present invention, and which does not destroy the pharmacological activity thereof and is nontoxic when administered in doses sufficient to deliver a therapeutic amount of the compositions of the present invention.

As used herein, the term “pharmaceutically acceptable salts” refers to salts prepared from pharmaceutically acceptable non-toxic bases including inorganic bases and organic bases. Salts derived from nonorganic bases include sodium, potassium, lithium, ammonia, calcium, magnesium, ferrous, zinc, manganous, aluminum, ferric, manganic salts and the like. Salts derived from pharmaceutically acceptable organic non-toxic bases include salts of primary, secondary, tertiary and quatemary amines, substituted amines including naturally occurring substituted amines, cyclic amines and basic ion exchange resins, such as triethylamine, tripropylamine, 2-dimethylaminoethanol, 2-diethylaminoethanol, lysine, arginine, histidine, caffeine, procaine, N-ethylpiperidine, hydrabamine, choline, betaine, ethylenediamine, glucosamine, methylglycamine, theobromine, purines, piperazine, piperidine, polyamine resins and the like.

As used herein, the term “prebiotic(s)” refers to a range of non-digestible dietary supplements which selectively stimulate the growth or activities, or both, of certain bacteria such as lactobacili or bifidobacteria in the colon. Non limiting examples of prebiotics include larch arabinogalactans, lactulose, lactitol, oligosaccharides and inulin.

As used herein, the term “probiotic(s)” refers to bacteria that assist in balancing the levels of indigenous microorganisms in the human body, especially the gastro-intestinal system, to promote healthy gut ecology and normal gastro-intestinal function. Probiotics participate in the regulation of intestinal functions such as mucous secretion and utilization, nutrient absorption, gastrointestinal motility, and splanchnic blood flow. (Clin Nutr 1996; 15). Probiotics also prevent the overgrowth of potentially pathogenic organisms and stimulate the intestinal immune defense system. (Clin Nutr 1996; 15). Non-limiting exemplary probiotics include, Bifidobacterium species such as Bifidobacterium bifidum; Bifidobacterium brevis, Bifidobacterium longus, Bifidobacterium infantis; Lactobacillus species, such as, Lactobacillus acidophilus, Lactobacillus bifidus, Lactobacillus brevis, Lactobacillus bulgaricus, Lactobacillus casei, Lactobacillus delbruekii, Lactobacillus lactis, Lactobacillus plantarum, Lactobacillus reuteri, Lactobacillus rhamnosus, Lactobacillus salivarius; Enterococcus faecium; Saccharomyces boulardii, and Streptococcus thermophilus.

As used herein, the term “substantially free” refers to a state of being wholly or almost wholly absent or lacking of a particular characteristic or compound.

As used herein, the term “substantially together,” refers to administering to a subject active ingredients in separate dosage forms, such that, the active ingredients can be administered either simultaneously or within a period of time such that the subject receives benefit of the aggregate effects of the separate dosage forms. For example, the active ingredients may be taken together or within a few seconds to at least about 30 minutes of one another.

In a preferred embodiment, anti-flatulent simethicone is selected on a basis for the therapeutic treatment of colic, including the treatment of bloating, crying, gas, cramping, regurgitation, diarrhea and gastrointestinal pain associated therewith. Simethicone is described as a mixture of fully methylated linear siloxane polymers containing repeating units of the formula [-(CH₃)₂SiO-]ₙ, stabilized with tremethylsiloxy end-blocking units of the formula [(CH₃)₃SiO-], and silicon dioxide. Simethicone or dimethylpolysiloxane is sometimes also referred to as polysiloxane or organopolysiloxane. Without intending to be bound by theory, simethicone is an anti-foaming agent which relieves flatulence by dispersing and preventing the formation of mucus-surrounding gas pockets in the gastro-intestinal tract. It is generally believed that simethicone acts in the stomach and intestines to change the surface tension of gas bubbles, enabling them to coalesce and be liberated more easily through belching or passing flatus.

In another preferred embodiment, the histamine H1-receptor antagonist diphenhydramine is selected on a basis for the therapeutic treatment of colic, including the treatment of bloating, crying, gas, cramping, regurgitation, diarrhea and gastrointestinal pain associated therewith. Diphenhydramine, a first-generation, non-selective antihistamine, binds non-selectively to central and peripheral H₁ receptors, resulting in sedation and CNS depression. Additionally, due to its cholinergic (atropine-like effect) actions, diphenhydramine has a drying effect by suppressing cholinergically innervated exocrine glands. It also possesses anti-emetic and anti-muscarinic effects as well, resulting in decreased nausea, motion sickness and vomiting.

The above antiflatulent and histamine H₁-receptor antagonist compounds can also be formulated, administered or ingested in combination with one or more prebiotic such as larch arabinogalactans and/or inulin. Larch arabinogalactans are polysaccharide powder derived from the wood of the larch tree (Larix species) and comprised of approximately ninety eight percent (98%) arabinogalactan. Larch arabinogalactan is approved by the U.S. Food and Drug Administration (FDA) as a source of dietary fiber, and is generally considered a safe, effective immune-stimulating agent for pediatric and adult use. Larch arabinogalactans are generally considered to be a substrate for the production of healthy bacteria in the gut of a host organism, thereby eliciting a clinically beneficial result on the host. Healthy gut ecology has been implicated in enhanced immune response, necessary for prevention of infection and overgrowth of yeast, fungi and pathogenic bacteria such as, H. pylori implicated in duodenal ulcer, Streptococcus species in otitis media, and Clostridium in infectious diarrhea. Additionally, larch arabinogalactan promotes healthy gut ecology by facilitating production of beneficial bacteria that is responsible for vitamin synthesis (e.g. folic acid), as well as production of essential medium-chain fatty acids.

Inulin is a natural storage carbohydrate found in numerous edible plant species including chicory, artichoke, leek, onion, asparagus, wheat, barley, rye, garlic, and bananas. Inulin belongs to the group of fructans. Fructans are compounds with one or more fructosyl-fructose linkages. Inulin is material with mostly or exclusively a β(2-1) fructosyl-fructose linkage. In most cases a glucose moiety can be found at the end of the fructose chain. Like larch arabinogalactans, inulin is generally considered to be a substrate for the production of healthy bacteria in the gut of a host. When digested, inulin travels through the digestive system essentially unchanged until reaching the large intestine where the colonic micro flora ferments it to produce various metabolic end products. Inulin may also be used as a sweetening agent.

The antiflatulent and histamine H₁-receptor antagonist compounds can also be formulated, administered or ingested in combination with probiotic bacteria. Probiotics work by colonizing the small intestine and crowding out disease-causing bacteria, thereby restoring balance to the intestinal flora. Some probiotics may also produce substances that inhibit pathogenic bacteria, compete for nutrients with pathogenic bacteria, and stimulate the body's own immune system. In breast-fed infants, bifidobacteria account for more than 90% of total intestinal bacteria, while in bottle-fed infants, bifidobacteria are not predominant. (Microbiol Immunol 1984;28). Certain strains of beneficial bacteria, including Lactobacilli and Bifidobacterium, appear to preferentially feed off of arabinogalactans or other prebiotic substrates. (J Food Microbiol 1994;24). Other probiotics, such as L. rhamnosus, L. plantarum, and L. reuteri, are often called and perceived “protective” gastrointestinal bacteria. (Clin Nutr 1996;15).

In addition, pharmaceutically acceptable carriers or adjuvants may be used in the pharmaceutical compositions of the present invention. Non-limiting exemplary examples of pharmaceutically acceptable carriers or adjuvants include, ion exchangers, alumina, aluminum stearate, lecithin, self-emulsifying drug delivery systems (SEDDS) such as d alpha-tocopherol polyethyleneglycol 1000 succinate, or other similar polymeric delivery matrices or systems, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-.beta.-cyclodextrins, or other solublized derivatives may also be advantageously used to enhance delivery of therapeutically-effective antiflatulents and histamine H₁-receptor antagonists with or without larch arabinogalactans of the present invention.

Moreover, the therapeutically-effective antiflatulent, histamine H₁-receptor antagonist compounds and larch arabinogalactans of the present invention may contain one or more asymmetric carbon atoms and thus may occur as racemates and racemic mixtures, single enantiomers, diastereomeric mixtures and individual diastereomers. Each stereogenic carbon may be of the R or S configuration. All such isomeric forms of these compounds are expressly included within the purview of the present invention.

The pharmaceutical compositions of the present invention can be conveniently prepared from, for example, commercially available antiflatulents, histamine H₁-receptor antagonists, larch arabinogalactans and probiotic, and may be formulated into liquid, suspension or solid dosage forms or combinations thereof. For example, the pharmaceutical composition may be formulated into a single unitary dose containing both the antiflatulent and the histamine H₁-receptor antagonist, and optionally, larch arabinogalactans and/or one or more probiotic in a liquid, suspension or solid dosage form. Likewise, the present invention contemplates formulating separate dosage forms of the ingredients and administering or ingesting the separate dosages substantially together, in the same or different dosage forms, such as, taking an antiflatulent as a liquid dose, a larch arabinogalactans as a suspension and a histamine H₁-receptor antagonist as a solid dose or combinations thereof, or taking or administering them separately as either solid, liquid, or suspension doses. Preferably a single unitary dose which comprises both or all of the active ingredients is formulated in an liquid dosage form such as a suspension.

When administering or taking the active ingredients substantially together, but separately in same or different dosage forms, the order in which they are administered or ingested is not critical. In other words, for example, the antiflatulent and the histamine H₁-receptor antagonist, and optionally, larch arabinogalacatans and/or one or more probiotic may be ingested simultaneously, or the antiflatulent may be ingested first followed by the histamine H₁-receptor antagonist and larch arabinogalactans, or the histamine H₁-receptor antagonist may be first ingested followed by larch arabinogalactans and the antiflatulent, or larch arabinogalactans may be first ingested followed by histamine H₁-receptor and antiflatulent. It is preferable, however to formulate the antiflatulent and the histamine H₁-receptor antagonist optionally with larch arabinogalactans and/or inulin and/or one or more probiotic into suspension mixtures which can be co-ingested as single unitary dosages on an as-needed basis, i.e., at or after the onset of “colicky” behavior.

Non-limiting examples of commercially available antiflatulents include, MYLICON® available from Johnson & Johnson—Merck Consumer Pharmaceuticals Co. of Ft. Washington, Pa., PHAZYME® available from GlaxoSmithKline.

A non-limiting examples of commercially available histamine H₁-receptor antagonists includes, BENADRYL® available from Pfizer, Inc. of 235 East 42nd Street New York, N.Y. 10017, USA.

There are many commercially available prebiotic and probiotic supplements.

As will be appreciated, the compositions of the present invention further comprise a pharmaceutically acceptable carrier or adjuvant in combination with at least one antiflatulent, at least one histamine H₁-receptor antagonist or a pharmaceutically acceptable salt thereof, and optionally, larch arabinogalactans. Further, the pharmaceutical methods of the present invention comprise a synergistic composition comprising an antiflatulent like simethicone, a histamine H₁ receptor antagonist like diphenhydramine, and optionally, larch arabinogalactans, wherein the active ingredients are present in effective amounts to provide relief from pain and/or discomfort associated with gastrointestinal disorders such as colic. Moreover, the dosing of the active ingredients is based on the weight and age of the subject being treated.

Simethicone, generally known to have a safe pediatric and adult profile is administered in an effective amount to substantially change the surface tension of gas bubbles. It is believed, without intending to be bound by theory, simethicone's reduction of cramping and gastrointestinal motility prevents “turbulence” that may be associated with gas production. The compositions of the present invention for the treatment of colic comprise formulations wherein an antiflatulent like simethicone is present in an amount ranging from about 40 mg/ml to about 120 mg/ml. More preferably, an antiflatulent is present in an amount of about 67 mg/ml.

In infantile colic, diphenhydramine is administered in a basal dosing regime to substantially inhibit respiratory, vascular and gastrointestinal smooth muscle constriction in infants and young children. Such basal levels of diphenhydramine reduce the risk of adverse side effects observed when higher doses of diphenhydramine are administered to infants and young children. The dosage is high enough to elicit a desired response in reduction of colic symptoms (anti-muscarinic) along with mild sedation (anticholinergic), but not high enough to affect bronchial airways with subsequent constriction of airways with possible loss of breathing. The compositions of the present invention for the treatment of colic comprise formulations wherein a histamine H₁ receptor antagonist like diphenhydramine is present in an amount ranging from about 1 mg/ml to about 4.0 mg/ml. More preferably, a histamine H₁ receptor antagonist is present in an amount of about 2.0 mg/ml.

Prebiotics, documented to have a safe pediatric and adult profile, are administered in an effective amount to increase immune-modulating activity in the gastro-intestinal tract. The compositions of the present invention for the treatment of colic optionally comprise one or more prebiotic such as larch arabinogalactans and/or inulin present in the amount from about 25 mg/ml to about 500 mg/ml. More preferably, one or more prebiotic is present in an amount of about 250 mg/ml.

One or more probiotic, generally known to have a safe pediatric and adult profile, is administered in an effective amount to promote healthy gut ecology and normalize gastro-intestinal function. The compositions of the present invention for the treatment of colic optionally comprise one or more probiotic present in an amount from about three million (3,000,000) to about thirty billion (30,000,000,000) colony forming units (CFU), or about 2.5 billion to 5 billion viable cells per dose or about 20 billion viable cells per day. More preferably, one or more probiotic is present in about three billion (3,000,000,000) CFU, which is about 100 mg/ml.

It is surprising that the combination of simethicone and diphenhydramine gives an effective result for the treatment of colic better than simethicone or diphenhydramine alone. It has also been surprisingly discovered that the present invention achieves effective results using less diphenhydramine which in turn reduces any safety risks associated with diphenhydramine alone.

Based on current pediatric recommendations for simethicone, the methods for the treatment of colic comprise administering and/or ingesting an antiflatulent like simethicone in a dosage amount of about 40 mg/0.6 ml to about 80 mg/1.2 ml per dose. More preferably, an antiflatulent such as simethicone is administered and/or ingested in an amount of about 40 mg/0.6 ml of three (3) to six (6) times daily. Most preferably, an antiflatulent like simethicone is administered and/or ingested in an amount of about 40 mg/0.6 ml four (4) times daily, about every four (4) to six (6) hours.

Methods for the treatment of colic include daily doses of histamine H₁-receptor antagonist, based on the current pediatric guidelines for diphenhydramine, which are about 5 mg/kg/day or about 150 mg/m₂/day for children over 10 kg. Since most children in need of therapeutic treatment of colic fall below this dosing guideline (i.e., less than 10 kg or 22 lbs), the amount of histamine H₁-receptor antagonist of the present invention is a lower but therapeutically useful dose of about 1.25 mg/kg/day. More preferably, a histamine H₁-receptor antagonist like diphenhydramine is administered and/or ingested in an amount from about 1.25 mg/0.6 ml to about 2.5 mg/1.2 ml three (3) to six (6) times daily. Most preferably, a histamine H₁-receptor antagonist like diphenhydramine is administered and/or ingested in an amount of about 1.25 mg/0.6 ml (i.e., about 1.25 mg/kg/day or about 0.5 mg/lb) four (4) times daily, about every four (4) to six (6) hours.

Methods for the treatment of colic optionally include a prebiotic such as, larch arabinogalactans and/or inulin, in an amount from about 125 mg/0.6 ml to about 250 g/1.2 ml per dose. More preferably, larch arabinogalactans is administered and/or ingested in an amount of about 125 g/0.6 ml three (3) to six (6) times daily. Most preferably, larch arabinogalactans is administered and/or ingested in an amount of about 125 g/0.6 ml four (4) times daily every four (4) to six (6) hours. Larch arabinogalactans are considered safe at all current dosing stratagems.

Methods for the treatment of colic also optionally include one or more probiotic in an amount of about 3 mg/0.6 ml to about 9 mg/0.6 ml per dose. More preferably, one or more probiotic is administered and/or ingested in an amount of about 6 mg/0.6 ml three (3) to six (6) times daily, and most preferably, four (4) times daily, about every four (4) to six (6) hours. Most preferably, one or more probiotic is administered and/or ingested in an amount of about 2.5 billion viable cells per 0.6 ml to about 5 billion viable cells per 1.2 ml three (3) to six (6) times daily, and most preferably, four times daily, about every four (4) to six (6) hours.

Moreover, the methods and compositions for the treatment of colic in infants and for gastrointestinal disorders in children and adults comprise administering/ingesting the active ingredients, an antiflatulent such as simethicone, a histamine H₁-receptor antagonist like diphenhydramine, and optionally, one or more prebiotic such as larch arabinogalactans and/or inulin, and/or one or more probiotic, based on the weight and age of the subject being treated (mg/kg/day). Practicing physicians will know how much of the active ingredients to administer based on methods known in the pharmaceutical arts, such as, Remington: The Science and Practice of Pharmacy, 20th Ed. 2000 Mack Publishing, and Martindale—The Complete Drug Reference, 33rd Ed. 1999, Pharmaceutical Press. For example, depending on the size and weight of an individual, simethicone is present and administered/ingested in an amount ranging from about 40 mg/ml to about 120 mg/ml; diphenhydramine is present in an amount ranging from about 1 mg/ml to about 5 mg/ml; one or more prebiotic is present in an amount ranging from about 250 mg/ml to about 1000 mg/ml; and one or more probiotic is present in an amount ranging from about 5 mg/ml to about 1000 mg/ml.

The pharmaceutical compositions may be in a form suitable for oral use, for example, as tablets, lozenges, aqueous or oily suspensions, dispersible powders or granules, emulsions, hard or soft capsules, syrups or elixirs. Compositions intended for oral use may be prepared according to any method known to the art for the manufacturer of pharmaceutical compositions and such compositions may contain one or more agents such as, for example, sweetening agents, flavoring agents, coloring agents and the like, in order to provide a pharmaceutically elegant and palatable preparation.

Tablets contain the active ingredients in admixture with nontoxic pharmaceutically acceptable excipients which are suitable for manufacture of tablets. These excipients may be, inert diluents, for example, calcium carbonate, sodium carbonate, lactose, calcium phosphate or sodium phosphate; granulating and disintegrating agents, for example, alginic acid, croscarmellose sodium, maize starch or; binding agents, for example, acacia, gelatine or starch, and lubricating agents, for example, magnesium stearate or stearic acid. The tablets may be uncoated or they may be coated by known techniques to delay disintegration and absorption in the gastro-intestinal tract and thereby provide an even longer sustained action over a period of time. The tablets may be chewable or non-chewable and designed to desired weight, potency and hardness through well known skills in the pharmaceutical arts.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with a suitable oil medium, for example, arachis oil, liquid paraffin or olive oil.

Formulations for oral use may also be presented as lozenges wherein the active ingredients are mixed into a hard candy composition. Suitable hard candy compositions can be made from varying, but highly concentrated, sucrose solutions including corn syrup as a second essential ingredient. Other known hard candy compositions may utilize any suitable good testing, sweet excipient other than sucrose.

Aqueous suspensions contain the active ingredients in admixture with excipients suitable for the manufacture of aqueous suspensions. Such excipients or combinations thereof may be suitable suspending agents, for example, alginates, carboxymethylcellulose, carboxypolymethylene, carrageenan, colloidal silcon dioxide, corn starch, flowable starch, gelatin, guar gum, gum acacia, gum tragacanth, hydroxypropylcellulose, hydroxypropylmethylcellulose, maltodextrin, methylcellulose, microcrystalline cellulose, pectin, polyethylene glycol 800, polyvinyl alcohol, polyvinylpyrrolidone, sodium alginate, sodium carboxymethyl cellulose or xanthum gum; dispersing or wetting agents may be any suitable naturally occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol, for example, polyoxyethylene sorbitol monnoleate, or condensation product of ethylene oxide with partial esters derived from fatty acids and hexitol and anhydrides, for example, polyoxyethelyne sobirtan monooleate, or water. The aqueous suspensions may also contain one or more suitable preservatives, for example, ethyl, or n-propyl, p-hydroxy benzoate, one or more suitable coloring agents, one or more suitable flavoring agents such as, cinnamon, chocolate, fruit flavors (i.e., cherry, grape, orange, strawberry, etc.), menthol, mints, vanilla and combination of two or more thereof, one or more suitable sweetening agents, such as calcium cyclamate, dextrose, fructose, galactose, glucose, glycerin, maltose, mannitol, mannose, ribose, partially hydrolyzed starch solids, partially hydrolyzed corn syrup solids, sodium cyclamate, sorbital, inulin, sucralose, sucrose, xylitol, or xylose, and one or more suitable coloring agents.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents may be exemplified by those already mentioned above. Additional suitable excipients, for example, sweetening, flavoring and coloring agents, may also be present.

Syrups and elixirs may be formulated with suitable sweetening agents, for example, one or more of glycerol, sorbitol, inulin, sucrose or xylose. Such formulations may also contain suitable demulcents, preservatives such as citric acid and flavoring and coloring agents.

In order to further illustrate the present invention and the advantages thereof, the following specific non-limiting Examples are given, it being understood that these Examples are intended only to be illustrations without serving as a limitation on the scope of the present invention.

### EXAMPLE 1

An oral pharmaceutical composition for the treatment of colic in infants, and for the treatment of gastrointestinal disorders in adults, is formulated in the following non-limiting examples.

| | Percent | | | |
|---|---|---|---|---|
| | Weight/volume | | Percent Weight/ | |
| Ingredient | (Pediatric) | | volume (Adult) | |
| Simethicone | 2 | g | 1920 | mg |
| Diphenhydramine | 62.5 | mg | 150 | mg |
| Microcrystalline cellulose | 1 | g | 1 | g |
| Croscarmellose sodium | 1 | gram | 1 | g |
| Citric acid | 50 | mg | 50 | mg |
| Xylose | 500 | mg | 2 | g |
| Sorbitol (70% w/w) | 5 | ml | 30 | ml |
| Natural flavor-non- | 2 | ml | 5 | ml |
| ETOH vanilla extract | | | | |

| | | |
|---|---|---|
| Purified water | q.s. to 30 ml | q.s. to 120 ml (4 oz) |

The above pediatric and adult formulations are prepared by known methods in the pharmaceutical arts.

The pediatric formulation provides a dose of the active ingredients, including, 40 mg/0.6 ml of simethicone and 1.25 mg/0.6 ml of diphenhydramine. A typical daily dosage amount for providing relief from pain and/or discomfort of colic in an infant weighing about 5 kg (11 lbs) is about 0.6 ml (about one half of a dropperful) four times a day, taken about every four (4) to six (6) hours. For infants weighing between about 5 and 10 kg (11 to 22 lbs), a typical dosage is about 1.2 mls (about 1 dropperful) four times a day, taken about every four (4) to six (6) hours.

The adult formulation provides a dose of the active ingredients for providing relief from pain and/or discomfort of gastrointestinal disorders in an adult weighing from about 50 kg to about 100 kg, including, from about 80 mg/5 ml (1 teaspoon) to about 160 mg/10 ml (2 teaspoons) of simethicone and from about 6.25 mg/5 ml (1 teaspoon) to about 12.5 mg/10 ml (2 teaspoons) of diphenhydramine, taken four times daily, about every four (4) to six (6) hours.

### EXAMPLE 2

An oral pharmaceutical composition for the treatment of colic in infants, and for the treatment of gastrointestinal disorders in adults, is formulated in the following non-limiting examples.

| | Percent | | Percent | |
|---|---|---|---|---|
| | Weight/volume | | Weight/volume | |
| Ingredient | (Pediatric) | | (Adult) | |
| Simethicone | 2 | g | 1920 | mg |
| Diphenhydramine | 62.5 | mg | 150 | mg |
| Larch arabinogalactans | 1 | g | 1.2 | g |
| Croscarmellose sodium | 1 | g | 1 | g |
| Microcrystalline cellulose | 1 | g | 1 | g |
| Xylose | 500 | mg | 2 | g |
| Citric acid | 50 | mg | 50 | mg |
| Sorbital (70% w/w) | 5 | ml | 30 | ml |
| Vanilla extract (non-ETOH) | 2 | ml | 5 | ml |

| | | |
|---|---|---|
| Purified water | q.s. to 30 ml | q.s. to 120 ml (4 oz) |

The above pediatric and adult formulations are prepared by known methods in the pharmaceutical arts.

The pediatric formulation provides a dose of the active ingredients, including, about 40 mg/0.6 ml of simethicone, about 1.25 mg/0.6 ml of diphenhydramine and about 20 mg/0.6 ml of larch arabinogalactans. A typical daily dosage amount for providing relief from pain and/or discomfort of colic in an infant weighing about 5 kg (11 lbs) is about 0.6 ml (about one half of a dropperful) four times a day, taken about every four (4) to six (6) hours. For infants weighing between about 5 and 10 kg (11 to 22 lbs), a typical dosage is about 1.2 mls (about 1 dropperful) four times a day, taken about every four (4) to six (6) hours.

The adult formulation provides a dose of the active ingredients for providing relief from pain and/or discomfort of gastrointestinal disorders in an adult weighing from about 50 kg to about 100 kg, including, from about 80 mg/5 ml (1 teaspoon) to about 160 mg/10 ml (2 teaspoons) of simethicone, from about 6.25 mg/5 ml (1 teaspoon) to about 12.5 mg/10 ml (2 teaspoons) of diphenhydramine, and from about 50 mg/5 ml to about 100 mg/10 ml of larch arabinogalactans, taken four times daily, about every four (4) to six (6) hours.

### EXAMPLE 3

An oral pharmaceutical composition for the treatment of colic in infants, and for the treatment of gastrointestinal disorders in adults, is formulated in the following non-limiting examples.

| | Percent | | Percent | |
|---|---|---|---|---|
| | Weight/volume | | Weight/volume | |
| Ingredient | (Pediatric) | | (Adult) | |
| Simethicone | 2 | g | 1920 | mg |
| Diphenhydramine | 62.5 | mg | 150 | mg |
| Probiotic Mixture | 10,000,000 | CFU | 20,000,000 | CFU |
| (L. acidophilus, L. | | | | |
| rhamnosus, L. reuteri plus | | | | |
| Bifidobacterium in | | | | |
| about equal parts). | | | | |
| Croscarmellose sodium | 1 | g | 1 | g |
| Microcrystalline cellulose | 1 | g | 1 | g |
| Xylose | 500 | mg | 2 | g |
| Citric acid | 50 | mg | 50 | mg |
| Sorbital (70% w/w) | 5 | ml | 30 | ml |
| Vanilla extract (non-ETOH) | 2 | ml | 5 | ml |

| | | |
|---|---|---|
| Purified water | q.s. to 30 ml | q.s. to 120 ml (4 oz) |

The above pediatric and adult formulations are prepared by known methods in the pharmaceutical arts.

The pediatric formulation provides a dose of the active ingredients, including, about 40 mg/0.6 ml of simethicone, about 1.25 mg/0.6 ml of diphenhydramine and about 6 mg/0.6 ml of a probiotic mixture. A typical daily dosage amount for providing relief from pain and/or discomfort of colic in an infant weighing about 5 kg (11 lbs) is about 0.6 ml (about one half of a dropperful) four times a day, taken about every four (4) to six (6) hours. For infants weighing between about 5 and 10 kg (11 to 22 lbs), a typical dosage is about 1.2 mls (about 1 dropperful) four times a day, taken about every four (4) to six (6) hours.

The adult formulation provides a dose of the active ingredients for providing relief from pain and/or discomfort of gastrointestinal disorders in an adult weighing from about 50 kg to about 100 kg, including, from about 80 mg/5 ml (1 teaspoon) to about 160 mg/10 ml (2 teaspoons) of simethicone, from about 6.25 mg/5 ml (1 teaspoon) to about 12.5 mg/10 ml (2 teaspoons) of diphenhydramine, and from about 8.3 mg/5 ml to about 16.6 mg/10 ml of a probiotic mixture, taken four times daily, about every four (4) to six (6) hours.

### EXAMPLE 4

An oral pharmaceutical composition for the treatment of colic in infants, and for the treatment of gastrointestinal disorders in adults, is formulated in the following non-limiting examples.

| | Percent | | Percent | |
|---|---|---|---|---|
| | Weight/volume | | Weight/volume | |
| Ingredient | (Pediatric) | | (Adult) | |
| Simethicone | 2 | g | 1920 | mg |
| Diphenhydramine | 62.5 | mg | 150 | mg |
| Larch arabinogalactans | 1 | g | 1.2 | g |
| Probiotic Mixture | 10,000,000 | CFU | 20,000,000 | CFU |
| (Lactobacillus acidophilus, | | | | |
| L. rhamnosus, L. reuteri | | | | |
| plus Bifidobacterium brevis, | | | | |
| B. longus, and B. infantis | | | | |
| in about equal parts) | | | | |
| Croscarmellose sodium | 1 | g | 1 | g |
| Microcrystalline cellulose | 1 | g | 1 | g |
| Xylose | 500 | mg | 2 | g |
| Citric acid | 50 | mg | 50 | mg |
| Sorbital (70% w/w) | 5 | ml | 30 | ml |
| Vanilla extract (non-ETOH) | 2 | ml | 5 | ml |

| | | |
|---|---|---|
| Purified water | q.s. to 30 ml | q.s. to 120 ml (4 oz) |

The above pediatric and adult formulations are prepared by known methods in the pharmaceutical arts.

The pediatric formulation provides a dose of the active ingredients, including, about 40 mg/0.6 ml of simethicone, about 1.25 mg/0.6 ml of diphenhydramine, about 20 mg/0.6 ml of larch arabinogalactans, and about 6 mg/0.6 ml of a probiotic mixture. A typical daily dosage amount for providing relief from pain and/or discomfort of colic in an infant weighing about 5 kg (11 lbs) is about 0.6 ml (about one half of a dropperful) four times a day, taken every four (4) to six (6) hours. For infants weighing between about 5 and 10 kg (11 to 22 lbs), a typical dosage is about 1.2 mls (about 1 dropperful) four times a day, taken about every four (4) to six (6) hours.

The adult formulation provides a dose of the active ingredients for providing relief from pain and/or discomfort of gastrointestinal disorders in an adult weighing from about 50 kg to about 100 kg, including, from about 80 mg/5 ml (1 teaspoon) to about 160 mg/10 ml (2 teaspoons) of simethicone, from about 6.25 mg/5 ml (1 teaspoon) to about 12.5 mg/10 ml (2 teaspoons) of diphenhydramine, from about 50 mg/5 ml to about 100 mg/10 ml of larch arabinogalactans, and from about 8.3 mg/5 ml to about 16.6 mg/10 ml of a probiotic mixture, taken four times daily, about every four (4) to six (6) hours.

### EXAMPLE 5

An oral pharmaceutical composition for the treatment of colic in infants, and for the treatment of gastrointestinal disorders in adults, is formulated in the following non-limiting examples.

| | Percent | | Percent | |
|---|---|---|---|---|
| | Weight/volume | | Weight/volume | |
| Ingredient | (Pediatric) | | (Adult) | |
| Simethicone | 2 | g | 4.8 | g |
| Diphenhydramine | 62.5 | mg | 300 | mg |
| Microcrystalline cellulose | 1 | g | 1 | g |
| Croscarmellose sodium | 1 | gram | 1 | g |
| Citric acid | 50 | mg | 50 | mg |
| Xylose | 500 | mg | 2 | g |
| Inulin (Jerusalem Artichoke | 250 mg per 1.2 ml | | 500 mg per 5 ml | |
| Powder)(sweetening agent) | dose or 6.250 g | | dose or 12 g | |
| | per 30 ml | | per 120 ml | |
| Natural flavor- non-ETOH | 2 | ml | 5 | ml |
| vanilla extract | | | | |

| | | |
|---|---|---|
| Purified water | q.s. to 30 ml | q.s. to 120 ml (4 oz) |

The above pediatric and adult formulations are prepared by known methods in the pharmaceutical arts.

The pediatric formulation provides a dose of the active ingredients, including, 40 mg/0.6 ml of simethicone and 1.25 mg/0.6 ml of diphenhydramine. A typical daily dosage amount for providing relief from pain and/or discomfort of colic in an infant weighing about 5 kg (11 lbs) is about 0.6 ml (about one half of a dropperful) four times a day, taken about every four (4) to six (6) hours. For infants weighing between about 5 and 10 kg (11 to 22 lbs), a typical dosage is about 1.2 mls (about 1 dropperful) four times a day, taken about every four (4) to six (6) hours.

The adult formulation provides a dose of the active ingredients for providing relief from pain and/or discomfort of gastrointestinal disorders in an adult weighing from about 50 kg to about 100 kg, including, from about 80 mg/5 ml (1 teaspoon) to about 160 mg/10 ml (2 teaspoons) of simethicone and from about 6.25 mg/5 ml (1 teaspoon) to about 12.5 mg/10 ml (2 teaspoons) of diphenhydramine, taken four times daily, about every four (4) to six (6) hours.

### EXAMPLE 6

An oral pharmaceutical composition for the treatment of colic in infants, and for the treatment of gastrointestinal disorders in adults, is formulated in the following non-limiting examples.

| | Percent | | Percent | |
|---|---|---|---|---|
| | Weight/volume | | Weight/volume | |
| Ingredient | (Pediatric) | | (Adult) | |
| Simethicone | 2 | g | 4.8 | g |
| Diphenhydramine | 62.5 | mg | 300 | mg |
| Larch arabinogalactans | 250 mg per 1.2 ml | | 500 mg per 5 ml | |
| | dose or 6.250 g | | dose or 12 g | |
| | per 30 ml bottle | | per 120 ml bottle | |
| Croscarmellose sodium | 1 | g | 1 | g |
| Microcrystalline cellulose | 1 | g | 1 | g |
| Xylose | 500 | mg | 2 | g |
| Citric acid | 50 | mg | 50 | mg |
| Inulin (Jerusalem Artichoke | 250 mg per 1.2 ml | | 500 mg per 5 ml | |
| Powder) Sweetening | dose or 6.250 g | | dose or 6.250 g | |
| agent | per 30 ml | | per 120 ml | |
| Vanilla extract (non-ETOH) | 2 | ml | 5 | ml |

| | | |
|---|---|---|
| Purified water | q.s. to 30 ml | q.s. to 120 ml (4 oz) |

The above pediatric and adult formulations are prepared by known methods in the pharmaceutical arts.

The pediatric formulation provides a dose of the active ingredients, including, about 40 mg/0.6 ml of simethicone, about 1.25 mg/0.6 ml of diphenhydramine and about 20 mg/0.6 ml of larch arabinogalactans. A typical daily dosage amount for providing relief from pain and/or discomfort of colic in an infant weighing about 5 kg (11 lbs) is about 0.6 ml (about one half of a dropperful) four times a day, taken about every four (4) to six (6) hours. For infants 745 weighing between about 5 and 10 kg (11 to 22 lbs), a typical dosage is about 1.2 mls (about 1 dropperful) four times a day, taken about every four (4) to six (6) hours.

The adult formulation provides a dose of the active ingredients for providing relief from pain and/or discomfort of gastrointestinal disorders in an adult weighing from about 50 kg to about 100 kg, including, from about 80 mg/5 ml (1 teaspoon) to about 160 mg/10 ml (2 teaspoons) of simethicone, from about 6.25 mg/5 ml (1 teaspoon) to about 12.5 mg/10 ml (2 teaspoons) of diphenhydramine, and from 100 mg/5 ml to about 2000 mg/10 ml of larch arabinogalactans, taken four times daily, about every four (4) to six (6) hours.

### EXAMPLE 7

An oral pharmaceutical composition for the treatment of colic in infants, and for the treatment of gastrointestinal disorders in adults, is formulated in the following non-limiting examples.

| | Percent | | Percent | |
|---|---|---|---|---|
| | Weight/volume | | Weight/volume | |
| Ingredient | (Pediatric) | | (Adult) | |
| Simethicone | 2 | g | 4.8 | g |
| Diphenhydramine | 62.5 | mg | 300 | mg |
| Probiotic Mixture in designated ratios: | 5 billion viable cells | | 10 billion viable cells | |
| (L. acidophilus (about 40%), L. | (CFU) per 1.2 ml (cc) or | | (CFU) per 5 ml or 240 | |
| rhamnosus, L. casei (LGG), and L. | 125 billion viable cells | | billion viable cells per | |
| plantarum (about 15% total), | per 30 ml bottle | | 120 ml (cc) bottle | |
| Bifidobacterium bifidum (about 35%), | | | | |
| Bifidobacerium brevis, Bifidobacterium | | | | |
| longus and Bifidobacterium infantis | | | | |
| (about 10% total), and Streptococcus | | | | |
| thermophilus (about 5%)). | | | | |
| Croscarmellose sodium | 1 | g | 1 | g |
| Microcrystalline cellulose | 1 | g | 1 | g |
| Xylose | 500 | mg | 2 | g |
| Citric acid | 50 | mg | 50 | mg |
| Inulin (Jerusalem Artichoke Powder) | 250 mg per 1.2 ml dose or | | 500 mg per 5 ml dose or | |
| (sweetening agent) | 6.250 g per 30 ml | | 12 g per 120 ml | |
| Vanilla extract (non-ETOH) | 2 | ml | 5 | ml |

| | | |
|---|---|---|
| Purified water | q.s. to 30 ml | q.s. to 120 ml (4 oz) |

The above pediatric and adult formulations are prepared by known methods in the pharmaceutical arts.

The pediatric formulation provides a dose of the active ingredients, including, about 40 mg/0.6 ml of simethicone, about 1.25 mg/0.6 ml of diphenhydramine and about 6 mg/0.6 ml of a probiotic mixture. A typical daily dosage amount for providing relief from pain and/or discomfort of colic in an infant weighing about 5 kg (11 lbs) is about 0.6 ml (about one half of a dropperful) four times a day, taken about every four (4) to six (6) hours. For infants weighing between about 5 and 10 kg (11 to 22 lbs), a typical dosage is about 1.2 mls (about 1 dropperful) four times a day, taken about every four (4) to six (6) hours.

The adult formulation provides a dose of the active ingredients for providing relief from pain and/or discomfort of gastrointestinal disorders in an adult weighing from about 50 kg to about 100 kg, including, from about 80 mg/5 ml (1 teaspoon) to about 160 mg/10 ml (2 teaspoons) of simethicone, from about 6.25 mg/5 ml (1 teaspoon) to about 12.5 mg/10 ml (2 teaspoons) of diphenhydramine, and from about 5 billion viable cells/5 ml to about 10 billion viable cells/10 ml of a probiotic mixture, taken four times daily, about every four (4) to six (6) hours.

### EXAMPLE 8

An oral pharmaceutical composition for the treatment of colic in infants, and for the treatment of gastrointestinal disorders in adults, is formulated in the following non-limiting examples.

| | Percent | | Percent | |
|---|---|---|---|---|
| | Weight/volume | | Weight/volume | |
| Ingredient | (Pediatric) | | (Adult) | |
| Simethicone | 2 | g | 4.8 | g |
| Diphenhydramine | 62.5 | mg | 300 | mg |
| Larch arabinogalactans | 250 mg per 1.2 ml dose or | | 500 mg per 5 ml dose or | |
| | 6.250 g per 30 ml | | 12 per 120 ml bottle | |
| Probiotic Mixture percentage ratios: | 5 billion viable cells | | 10 billion viable cells | |
| (Lactobacillus acidophilus (about 40%), | (CFUs) per 1.2 ml dose or | | (CFUS) per 5 ml dose or | |
| L. rhamnosus, L. casei (LGG) and L. | 125 billion viable cells per | | 240 billion viable cells | |
| plantarum (about 15% total), | 30 ml bottle | | per 120 ml bottle | |
| Bifidobacterium bifidum (about 30%), B. | | | | |
| brevis, B. longus and B. infantis(about | | | | |
| 10% total), and Streptococcus | | | | |
| thermophilus (about 5%)) | | | | |
| Croscarmellose sodium | 1 | g | 1 | g |
| Microcrystalline cellulose | 1 | g | 1 | g |
| Xylose | 500 | mg | 2 | g |
| Citric acid | 50 | mg | 50 | mg |
| Inulin (as Jerusalem Artichoke powder) | 250 mg per 1.2 ml dose or | | 500 mg per 5 ml dose or | |
| sweetening agent | 6.250 g per 30 ml | | 12 g per 120 ml | |
| Vanilla extract (non-ETOH) | 2 | ml | 5 | ml |

| | | |
|---|---|---|
| Purified water | q.s. to 30 ml | q.s. to 120 ml (4 oz) |

The above pediatric and adult formulations are prepared by known methods in the pharmaceutical arts.

The pediatric formulation provides a dose of the active ingredients, including, about 40 mg/0.6 ml of simethicone, about 1.25 mg/0.6 ml of diphenhydramine, about 20 mg/0.6 ml of larch arabinogalactans, and about 6 mg/0.6 ml of a probiotic mixture. A typical daily dosage amount for providing relief from pain and/or discomfort of colic in an infant weighing about 5 kg (11 lbs) is about 0.6 ml (about one half of a dropperful) four times a day, taken every four (4) to six (6) hours. For infants weighing between about 5 and 10 kg (11 to 22 lbs), a typical dosage is about 1.2 mls (about 1 dropperful) four times a day, taken about every four (4) to six (6) hours.

The adult formulation provides a dose of the active ingredients for providing relief from pain and/or discomfort of gastrointestinal disorders in an adult weighing from about 50 kg to about 100 kg, including, from about 80 mg/5 ml (1 teaspoon) to about 160 mg/10 ml (2 teaspoons) of simethicone, from about 6.25 mg/5 ml (1 teaspoon) to about 12.5 mg/10 ml (2 teaspoons) of diphenhydramine, from about 100 mg/5 ml to about 2000 mg/10 ml of larch arabinogalactans, and from about 5 billion viable cells/5 ml to about 30 billion viable cells/10 ml of a probiotic mixture, taken four times daily, about every four (4) to six (6) hours.

### EXAMPLE 9

MYLICON™ brand simethicone was obtained from J&J-Merck. BENADRYL® brand diphenhydramine was obtained from Park-Davis (n/k/a Pfizer). An infant with colic was administered about 40 mg/0.6 ml of MYLICON® substantially together with about 1.25 mg/0.6 ml to about 2.5 mg/1.2 ml of BENADRYL® four times daily during episodes of colic from about one month in age to about six (6) months in age. The infant experienced significant improvement after drug therapy, both with diminution of colic symptoms (e.g., gas, cramping, vomiting), as well as, providing rest and reduction of crying and fussing episodes about 75% of the time. The parents of the treated infant stated that the treatment led to rest of the infant as well as for the parents, allowing resumption of other necessary daily activities. In addition the parents and doctor of the treated infant did not observe any adverse side effects.

Although illustrative embodiments of the present invention have been described in detail, it is to be understood that the present invention is not limited to those precise embodiments, and that various changes and modifications can be effected therein by one skilled in the art without departing from the scope and spirit of the invention as defined by the appended claims.

What is claimed is:

1. A pharmaceutical composition for the treatment of gastrointestinal disorders comprising:
a. one or more antiflatulent;
b. one or more competitive reversible histamine H₁-receptor antagonist; and
c. a pharmaceutically acceptable carrier.

2. The pharmaceutical composition of claim 1 further comprising one or more prebiotic.

3. The pharmaceutical composition of claim 1 further comprising one or more probiotic.

4. The pharmaceutical composition of claim 1, wherein the gastrointestinal disorder is selected from the group consisting of acid indigestion, colic, diarrhea, heartburn, irritable bowel syndrome, sour stomach, gas associated with the foregoing conditions, peptic ulcer disease of the esophagus, stomach or duodeum, indigestion, flatulence, dyspepsia of unknown origin including cancer of the stomach, infiltrative disease of the stomach including lymphoma, Crohn's disease, eosinophilic granuloma, tuberculosis, syphilis and sarcoidosis, abdominal lesions, chronic pancreatis, bilary disease, Zollinger-Ellison syndrome, motion sickness, and otitis media.

5. A method for the treatment of gastrointestinal disorders comprising administering to a subject in need thereof, a therapeutically effective dose of the composition of claim 1.

6. The method of claim 5 wherein the gastrointestinal disorders are selected from the group consisting of acid indigestion, colic, diarrhea, heartburn, irritable bowel syndrome, sour stomach, gas associated with the foregoing conditions, peptic ulcer disease of the esophagus, stomach or duodeum, indigestion, flatulence, dyspepsia of unknown origin including cancer of the stomach, infiltrative disease of the stomach including lymphoma, Crohn's disease, eosinophilic granuloma, tuberculosis, syphilis and sarcoidosis, abdominal lesions, chronic pancreatis, bilary disease, Zollinger-Ellison syndrome, motion sickness, and otitis media.

7. A pharmaceutical composition for the treatment of colic comprising:
a. one or more antiflatulent;
b. one or more competitive histamine Hi-receptor antagonist; and
c. a pharmaceutically acceptable carrier.

8. The pharmaceutical composition of claim 7, wherein the antiflatulent is selected from the group consisting of maltodextrin and organopolysiloxanes.

9. The pharmaceutical composition of claim 8, wherein the antiflatulent is an organopolysiloxane selected from the group consisting of dimethicone, dimethylpolysiloxane, methylpolysiloxane and simethicone.

10. The pharmaceutical composition of claim 9, wherein the oragnopolysiloxane is simethicone.

11. The pharmaceutical composition of claim 10, wherein the simethicone is present in an amount from about 40 mg/ml to about 120 mg/ml.

12. The pharmaceutical composition of claim 11, wherein the simethicone is present in an amount of about 80 mg/ml.

13. The pharmaceutical composition of claim 7, wherein in the histamine H₁-receptor antagonist is selected from the group consisting of acrivastine, astemizole, azatadine, azclastine, bromodiphenhydramine, brompheniramine, cetirizine, chlorpheniramine, clematine, cyproheptatine, desloratadine, dexbrompheniramine, dexchlorpheniramine, diphehydramine, doxylamine, fexofenadine, hydroxyzine, ketoffen, loratidine, norastemizole, phenindamine, pyrilamine, temelastine, terfenadine, tripelennamine and triprolidine.

14. The pharmaceutical composition of claim 13, wherein the histamine H₁-receptor antagonist is diphenhydramine.

15. The pharmaceutical composition of claim 14, wherein the diphenhydramine is present in an amount from about 1.0 mg/ml to about 4.0 mg/ml.

16. The pharmaceutical composition of claim 15, wherein the diphenhydramine is present in an amount of about 2.0 mg/ml.

17. The pharmaceutical composition of claim 7 further comprising one or more prebiotic.

18. The pharmaceutical composition of claim 17 wherein the prebiotic is selected from the group consisting of larch arabinogalactans, lactulose, lactitol, oligosaccharides and inulin.

19. The pharmaceutical composition of claim 18 wherein the prebiotic is larch arabinogalactans.

20. The pharmaceutical composition of claim 19, wherein the larch arabinogalactans are present in an amount from about 25 mg/ml to about 500 mg/ml.

21. The pharmaceutical composition of claim 20, wherein the larch arabinogalactans are present in an amount of about 250 mg/ml.

22. The pharmaceutical composition of claim 7 further comprising one or more probiotic selected from the group consisting of Bifidobacterium species such as Bifidobacterium bifidum; Bifidobacterium brevis, Bifidobacterium longus, Bifidobacterium infantis; Lactobacillus species, such as, Lactobacillus acidophilus, Lactobacillus bifidus, Lactobacillus brevis, Lactobacillus bulgaricus, Lactobacillus casei, Lactobacillus delbruekii, Lactobacillus lactis, Lactobacillus plantarum, Lactobacillus reuteri, Lactobacillus rhamnosus, Lactobacillus salivarius, Enterococcus faecium; Saccharomyces boulardii; and Streptococcus thermphilus.

23. The pharmaceutical composition of claim 7, wherein the composition is substantially free of dyes, alcohols, artificial flavors, artificial sweeteners and artificial preservatives.

24. The pharmaceutical composition of claim 7, wherein the pharmaceutical composition is in a liquid dosage form.

25. The pharmaceutical composition of claim 24, wherein the liquid dosage form is a suspension.

26. The pharmaceutical composition of claim 7, wherein the composition is in a solid dosage form.

27. A method for the treatment of colic comprising administering to a subject in need thereof one or more antiflatulent and one or more competitive histamine HI-receptor antagonist.

28. The method according to claim 27, wherein the antiflatulent is selected from the group consisting of maltodextrin and organopolysiloxanes.

29. The method according to claim 28, wherein the antiflatulent is an organopolysiloxane selected from the group consisting of, dimethicone, dimethylpolysiloxone, methylpolysiloxone and simethicone.

30. The method according to claim 29, wherein the organopolysiloxane is simethicone.

31. The method according to claim 30, wherein the simethicone is administered three (3) to six (6) times daily in an amount from about 25 mg/0.6 ml to about 50 mg/0.6 ml.

32. The method according to claim 31, wherein the simethicone is administered four times daily in an amount of about 40 mg/0.6 ml.

33. The method according to claim 27, wherein the histamine H₁-receptor antagonist is selected from the group consisting of acrivastine, astemizole, azatadine, azclastine, bromodiphenhydramine, brompheniramine, cetirizine, chlorpheniramine, clematine, cyproheptatine, desloratadine, dexbrompheniramine, dexchlorpheniramine, diphehydramine, doxylamine, fexofenadine, hydroxyzine, ketoffen, loratidine, norastemizole, phenindamine, pyrilamine, temelastine, terfenadine, tripelennamine and triprolidine.

34. The method according to claim 33, wherein the histamine H₁-receptor antagonist is diphenhydramine.

35. The method according to claim 34, wherein the diphenhydramine is administered three (3) to six (6) times daily in an amount from about 0.50 mg/0.6 ml to about 2.0 mg/0.6 ml.

36. The method according to claim 35, wherein the diphenhydramine is administered four (4) times daily in an amount of about 1.25 mg/0.6 ml.

37. The method according to claim 27 further comprising administering one or more prebiotic selected from the group consisting of larch arabinogalactans, lactulose, lactitol, oligosaccharides and inulin.

38. The method according to claim 37 wherein the prebiotic is larch arabinogalactans.

39. The method according to claim 38, wherein the larch arabinogalactans is administered three (3) to six (6) times daily in an amount of about 100 mg/0.6 ml to about 250 mg/0.6 ml.

40. The method according to claim 39, wherein the larch arabinogalactans is administered four times daily in an amount of about 125 mg/0.6 ml.

41. The method according to claim 27 further comprising administering one or more probiotic selected from the group consisting of Bifidobacterium species such as Bifidobacterium bifidum, Bifidobacterium brevis, Bifidobacterium longus, Bifidobacterium infantis; Lactobacillus species, such as, Lactobacillus acidophilus, Lactobacillus bifidus, Lactobacillus brevis, Lactobacillus bulgaricus, Lactobacillus casei, Lactobacillus delbruekii, Lactobacillus lactis, Lactobacillus plantarum, Lactobacillus reuteri, Lactobacillus rhamnosus, Lactobacillus salivarius; Enterococcus faecium; Saccharomyces boulardii; and Streptococcus thermophilus.
